(19) United States
(12) Patent Application Publication
(10) Pub. No.: US 2017/0119342 A1
(43) Pub. Date: May 4, 2017

(54) ULTRASONIC DEVICE, ULTRASONIC PROBE, AND ULTRASONIC IMAGING APPARATUS

(71) Applicant: SEIKO EPSON CORPORATION, Tokyo (JP)

(72) Inventor: Jiro TSURUNO, Okaya-shi (JP)

(73) Assignee: SEIKO EPSON CORPORATION, Tokyo (JP)

(21) Appl. No.: 15/297,296

(22) Filed: Oct. 19, 2016

(30) Foreign Application Priority Data
Oct. 29, 2015 (JP) 2015-212627

(51) Int. Cl. A61B 8/08; A61B 8/00

(52) U.S. Cl. CPC A61B 8/0841; A61B 8/463; A61B 8/5246

(57) ABSTRACT

An ultrasonic device has ultrasonic elements to transmit and receive ultrasonic waves. The ultrasonic device includes a first ultrasonic element array in which ultrasonic elements forming one channel among the ultrasonic elements are arranged in a first direction (scan direction) and a second ultrasonic element array in which ultrasonic elements forming one channel are arranged in the first direction. The second ultrasonic element array is disposed so as to be shifted from the first ultrasonic element array in a second direction (slice direction) crossing the first direction.

This application claims the benefit of Japanese Patent Application No. 2015-212627, filed on Oct. 29, 2015. The content of the aforementioned application is hereby incorporated by reference in its entirety.

### BACKGROUND

1. Technical Field

The present invention relates to an ultrasonic device, an ultrasonic probe including an ultrasonic device, and an ultrasonic imaging apparatus including an ultrasonic probe.

2. Related Art

A known ultrasonic device is configured to include a piezoelectric member, a backing unit, an acoustic matching layer, an acoustic lens, and the like. Ultrasonic waves generated by the piezoelectric member of the ultrasonic device are incident on a subject through the acoustic matching layer and the acoustic lens. Then, the ultrasonic device receives reflected waves (ultrasonic echoes) that are reflected from the inside of the subject, and generates a voltage corresponding to the strength of the reflected waves,

In the case of performing an insertion operation using a probe including such an ultrasonic device and an ultrasonic imaging apparatus, in order not to lose the sight of a needle tip, a needle is moved forward while tilting the probe so that the needle tip always overlaps the plane of the scanning lines of an ultrasonic image. In addition, there is a method of performing an insertion operation while recognizing the needle tip by sweeping the probe on the body surface of the subject so that blood vessels and the needle tip are always reflected in the image.

JP-A-2012-192162 discloses an ultrasonic diagnostic apparatus capable of improving the visibility of an insertion needle in order to increase the strength of the ultrasonic echo signal from the insertion needle.

Tilting or sweeping the probe in order to always reflect the needle tip in the image as described above requires a technique for operating the probe since it is difficult to hold the position of the moved probe and a probe contact position is not stable. For this reason, there has been a problem that the sight of the needle tip is likely to be lost since it is difficult to always reflect the needle tip in an image by moving the probe.

For this reason, an ultrasonic device, an ultrasonic probe, and an ultrasonic imaging apparatus capable of easily capturing the needle tip without moving the probe have been requested.

### SUMMARY

An advantage of some aspects of the invention is to solve at least a part of the problems described above, and the invention can be implemented as the following aspects or application examples.

### APPLICATION EXAMPLE 1

An ultrasonic device according to this application example is an ultrasonic device having ultrasonic elements to transmit and receive ultrasonic waves and including: a first ultrasonic element array in which ultrasonic elements forming one channel among the ultrasonic elements are arranged in a first direction; and a second ultrasonic element array in which ultrasonic elements forming one channel are arranged in the first direction. The second ultrasonic element array is disposed so as to be shifted from the first ultrasonic element array in a second direction crossing the first direction.

According to such an ultrasonic device, by driving the first and second ultrasonic element arrays separately, each of the first and second ultrasonic element arrays can emit ultrasonic waves. In addition, it is possible to receive an ultrasonic echo for generating an image corresponding to the ultrasonic element array along the second direction. Therefore, in the case of performing an insertion operation, by generating an image based on the received ultrasonic echo and moving the needle tip in the second direction, it is possible to reflect the needle tip in the image. As a result, it is possible to realize an ultrasonic device capable of easily capturing the needle tip without moving the ultrasonic device.

### APPLICATION EXAMPLE 2

In the ultrasonic device according to the application example, it is preferable that the first and second ultrasonic element arrays transmit and receive the ultrasonic waves by driving the first and second ultrasonic element arrays in combination in addition to driving the first and second ultrasonic element arrays separately.

According to such an ultrasonic device, by driving the ultrasonic element arrays in combination in addition to driving the ultrasonic element arrays separately, it is possible to receive ultrasonic echoes for generating a larger number of images than the number of ultrasonic element arrays. As a result, it is possible to realize an ultrasonic device capable of capturing the needle tip more easily without moving the ultrasonic device.

### APPLICATION EXAMPLE 3

In the ultrasonic device according to the application example, it is preferable that the first and second ultrasonic element arrays transmit and receive the ultrasonic waves by driving all of the ultrasonic elements of the first and second ultrasonic element arrays in addition to driving the first and second ultrasonic element arrays separately.

According to such an ultrasonic device, by driving all of the ultrasonic elements in addition to driving the ultrasonic element arrays separately, it is possible to receive ultrasonic echoes for generating a larger number of images than the number of ultrasonic element arrays. As a result, it is possible to realize an ultrasonic device capable of capturing the needle tip more easily without moving the ultrasonic device.

### APPLICATION EXAMPLE 4

In the ultrasonic device according to the application example, it is preferable to further include an acoustic unit in contact with a subject, and it is preferable that the acoustic unit includes a flat surface portion in contact with the subject.

According to such an ultrasonic device, since the acoustic unit includes a flat surface portion in contact with the subject, ultrasonic waves emitted from the ultrasonic elements formed in an array are converged by the acoustic unit in the case of driving the ultrasonic element arrays. Therefore, it is possible to receive an ultrasonic echo through which the inside of the subject including the needle tip can be captured.

### APPLICATION EXAMPLE 5

An ultrasonic probe according to this application example includes: the ultrasonic device according to any one of the above application examples; and a housing member that houses the ultrasonic device such that apart of the ultrasonic device is exposed.

According to such an ultrasonic probe, an ultrasonic probe is formed by housing the ultrasonic device including the first and second ultrasonic element arrays in the housing member. Therefore, it is possible to realize an ultrasonic probe capable of easily capturing the needle tip without moving the ultrasonic probe.

### APPLICATION EXAMPLE 6

An ultrasonic imaging apparatus according to this application example includes: the ultrasonic probe according to the application example; a processing device that controls the ultrasonic probe and generates an image in the ultrasonic element array based on an input signal from the ultrasonic probe; and a display device that displays the image generated by the processing device.

According to such an ultrasonic imaging apparatus, it is possible to generate a plurality of images in the second direction by driving a plurality of ultrasonic element arrays without moving the ultrasonic probe. Therefore, it is possible to realize an ultrasonic imaging apparatus capable of easily capturing the needle tip.

### BRIEF DESCRIPTION OF THE DRAWINGS

The invention will be described with reference to the accompanying drawings, wherein like numbers reference like elements.

FIG. 1 is a perspective view showing the schematic configuration of an ultrasonic imaging apparatus according to a first embodiment.

FIG. 2 is a perspective view showing the schematic configuration of an ultrasonic probe.

FIG. 3 is a perspective view showing the schematic configuration of an ultrasonic device.

FIG. 4 is a circuit block diagram of an ultrasonic imaging apparatus.

FIG. 5 is a plan view showing the schematic configuration of an ultrasonic element.

FIG. 6 is a sectional view showing the schematic configuration of an ultrasonic element.

FIG. 7 is an explanatory view showing the schematic configuration of an ultrasonic element array.

FIG. 8A is a schematic diagram showing a sectional image obtained in the case of driving an ultrasonic element array.

FIG. 8B is a schematic diagram showing a sectional image obtained in the case of driving an ultrasonic element array.

FIG. 8C is a schematic diagram showing a sectional image obtained in the case of driving all of two ultrasonic element arrays.

FIG. 9 is a schematic diagram showing the positional relationship among an insertion needle, a blood vessel, an ultrasonic probe, and sectional images in the case of performing an insertion operation.

FIG. 10 is a sectional image schematically showing the state of an insertion needle at the time of insertion operation.

FIG. 11 is a perspective view showing the schematic configuration of an ultrasonic device according to a second embodiment.

### DESCRIPTION OF EXEMPLARY EMBODIMENTS

### First Embodiment

In the present embodiment, an ultrasonic device 1, an ultrasonic probe 100 including the ultrasonic device 1, and an ultrasonic imaging apparatus 120 as an electronic apparatus including the ultrasonic probe 100 will be described with reference to the accompanying diagrams. In each diagram, the scale of each member is adjusted in order to have a recognizable size.

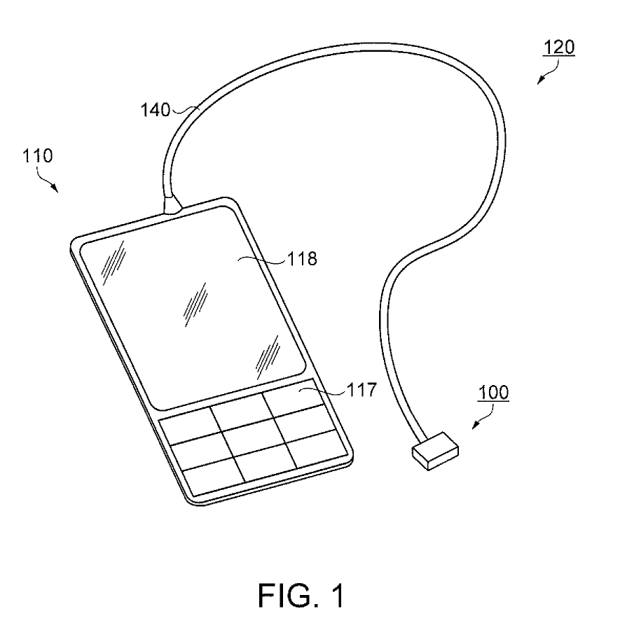

FIG. 1 is a perspective view showing the schematic configuration of the ultrasonic imaging apparatus 120 according to the first embodiment. The configuration of the ultrasonic imaging apparatus 120 will be described with reference to FIG. 1.

The ultrasonic imaging apparatus 120 of the present embodiment is an apparatus that holds the ultrasonic probe 100 on the body surface of a subject or the like so as to be in close contact with each other, transmits ultrasonic waves from the ultrasonic probe 100, receives reflected waves (ultrasonic echoes) that are reflected from the inside of the subject, analyzes data of the received ultrasonic echoes, and displays the data as an image. An operator performs an insertion operation or the like while checking the image.

The ultrasonic imaging apparatus 120 includes the ultrasonic probe 100 and a processing device 110. The processing device 110 is configured to include an operating unit 117, a display device 118, and the like. The ultrasonic probe 100 and the processing device 110 are connected to each other through a flexible cable 140, so that an electrical signal is transmitted or received therebetween. The display device 118 displays an image generated by the processing of the processing device 110 (image based on an ultrasonic echo detected by the ultrasonic probe 100). The operating unit 117 is configured to include key switches, and outputs a command based on the operation of the key switches to the processing device 110 (main control unit 115 to be described later).

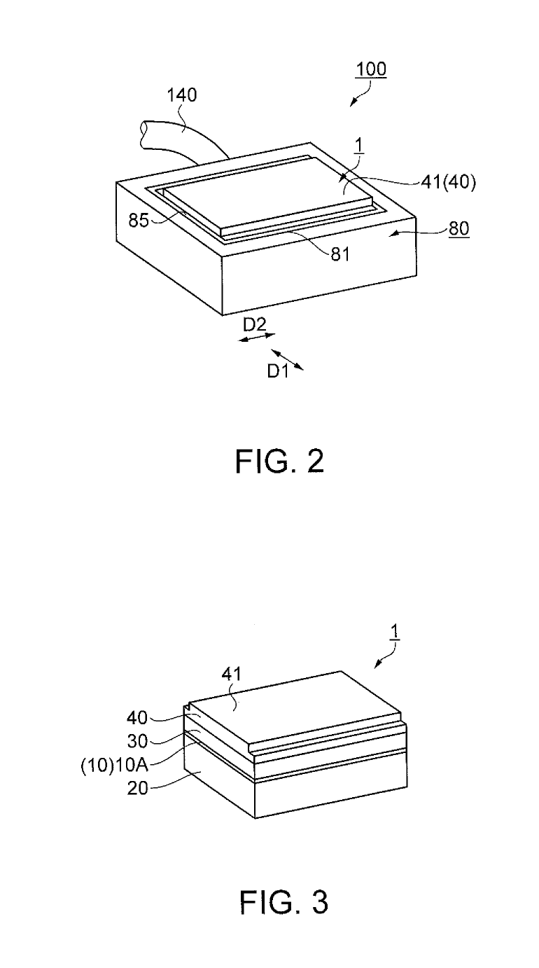

FIG. 2 is a perspective view showing the schematic configuration of the ultrasonic probe 100. Specifically, FIG. 2 is a perspective view when the ultrasonic probe 100 is viewed from a side on which the ultrasonic probe 100 is in close contact with the body surface. FIG. 3 is a perspective view showing the schematic configuration of the ultrasonic device 1. The configurations of the ultrasonic probe 100 and the ultrasonic device 1 will be described with reference to FIGS. 2 and 3.

As shown in. FIG. 2, the ultrasonic probe 100 of the present embodiment is configured to include the ultrasonic device 1, a housing member 80, and the like. The ultrasonic device 1 is generally formed in the shape of a rectangular flat plate, as shown in FIG. 3. Similar to the ultrasonic device 1, the housing member 80 is also formed in the shape of a rectangular flat plate. The housing member 80 has a housing unit 81, and houses the ultrasonic device 1 therein in a state in which an acoustic unit 40 (flat surface portion 41), which is a part of the ultrasonic device 1, is exposed. When housing the ultrasonic device 1 in the housing unit 81, a gap between the housing unit 81 and the ultrasonic device 1 is sealed by inserting a silicone-based sealing member 85 to a gap between the inner surface of the housing unit 81 and the outer surface of the ultrasonic device 1. The housing member 80 is formed using a synthetic resin member in the present embodiment. However, other members, for example, a metal member can be used without being limited thereto.

As shown in FIG. 3, the ultrasonic device 1 of the present embodiment is configured to include an acoustic matching layer 30, the acoustic unit 40, and a backing unit 20 that are provided above and below an ultrasonic element array 10A (ultrasonic element 10) formed in a rectangular shape. Ultrasonic waves generated by the ultrasonic element 10 of the ultrasonic device 1 are incident on a subject through the acoustic matching layer 30 and the acoustic unit 40. Then, the ultrasonic device 1 receives reflected waves of the ultrasonic waves (ultrasonic echoes) that are reflected from the inside of the subject, and generates a voltage corresponding to the strength of the echoes.

The acoustic matching layer 30 performs acoustic matching for making ultrasonic waves incident on the inside of the subject efficiently by suppressing the reflection of the ultrasonic waves by reducing the difference in acoustic impedance between the ultrasonic element array 10A and the subject.

As shown in FIGS. 2 and 3, the acoustic unit 40 includes the flat surface portion 41 having a flat surface that is an outer surface. By the flat surface portion 41 of the acoustic unit 40, the spread of ultrasonic waves emitted from the ultrasonic element array 10A converges. The ultrasonic element 10 of the present embodiment is a so-called thin film piezoelectric ultrasonic element. The ultrasonic elements 10 are disposed in an array to form the ultrasonic element array 10A. In a case where ultrasonic waves are emitted from the ultrasonic element array 10A, ultrasonic waves emitted from the flat surface portion 41 have a convergence characteristic up to a certain distance even in a case where a portion of the acoustic unit 40 in contact with the subject is not a lens but the flat surface portion 41 as in the present embodiment.

The backing unit 20 attenuates unnecessary ultrasonic waves emitted from the ultrasonic element array 10A, thereby improving the distance resolution in an image.

In the present embodiment, the ultrasonic element array 10A is configured to include two ultrasonic element arrays. Specifically, the ultrasonic element array 10A is configured to include an ultrasonic element array A and an ultrasonic element array B. The ultrasonic element array A and the ultrasonic element array B will be described in detail later.

As shown in FIG. 2, a scan direction D2 is defined as a direction parallel to the longitudinal direction of the acoustic unit 40, and a slice direction D1 is defined as a direction that is perpendicular to the longitudinal direction of the acoustic unit 40 and is parallel to the surface of the housing member 80 in which the housing unit 81 is formed. The scan direction D2 and the slice direction D1 are perpendicular to each other within this plane.

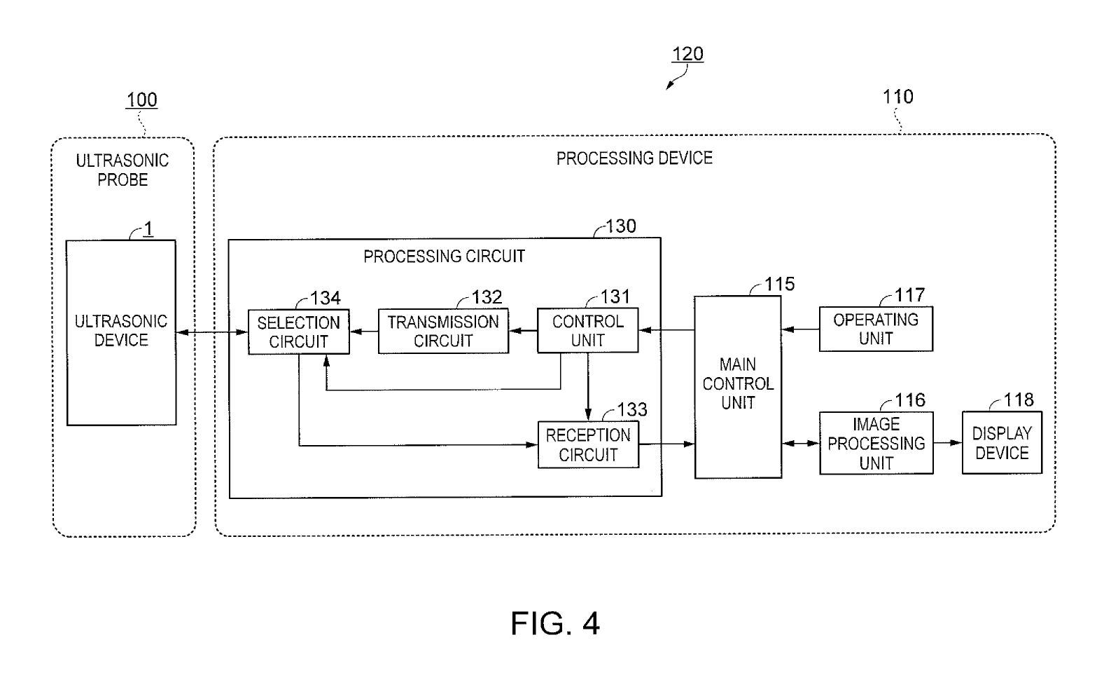

FIG. 4 is a circuit block diagram of the ultrasonic imaging apparatus 120. The circuit configuration of the ultrasonic imaging apparatus 120 will be described with reference to FIG. 4.

The ultrasonic imaging apparatus 120 includes the ultrasonic probe 100 and the processing device 110 as described above. The ultrasonic probe 100 includes the ultrasonic device 1 and the like. The processing device 110 includes a processing circuit 130, the main control unit 115, an image processing unit 116, the operating unit 117, the display device 118, and the like.

The processing circuit 130 includes a control unit 131, a transmission circuit 132, a reception circuit 133, a selection circuit 134, and the like. The processing circuit 130 performs transmission processing and reception processing of the ultrasonic device 1. The transmission circuit 132 outputs a transmission signal VT to the ultrasonic device 1 through the selection circuit 134 in a transmission period. Specifically, the transmission circuit 132 generates the transmission signal VT based on the control of the control unit 131, and outputs the transmission signal VT to the selection circuit 134. The selection circuit 134 outputs the transmission signal VT from the transmission circuit 132 based on the control of the control unit 131. The frequency and the amplitude voltage of the transmission signal VT can be set by the control unit 131.

The reception circuit 133 performs reception processing of a reception signal VR from the ultrasonic device 1. Specifically, in a reception period, the reception circuit 133 receives the reception signal VR from the ultrasonic device 1 through the selection circuit 134, and performs reception processing, such as amplification of a reception signal, gain setting, frequency setting, and A/D conversion (analog/digital conversion). The result of the reception processing is finally output to the image processing unit 116 as detection data (detection information). The reception circuit 133 can be configured to include, for example, a low noise amplifier, a voltage controlled attenuator, a programmable gain amplifier, a low pass filter, and an A/D converter.

The control unit 131 controls the transmission circuit 132 and the reception circuit 133. Specifically, the control unit 131 controls the generation and output processing of the transmission signal VT for the transmission circuit 132, and performs control of frequency setting, gain setting, or the like of the reception signal. VR for the reception circuit 133.

Based on the control of the control unit 131, the selection circuit 134 performs switching between the ultrasonic element arrays A and B to be driven, and outputs the transmission signal VT to the corresponding ultrasonic element arrays A and B. In the present embodiment, since the ultrasonic device 1 is driven using a so-called linear scan method, the selection circuit 134 has a function of sequentially switching a channel to be driven at a predetermined timing.

The main control unit 115 controls transmission and reception of ultrasonic waves for the ultrasonic probe 100, and controls image processing of detection data for the image processing unit 116. The image processing unit 116 receives the detection data from the reception circuit 133, and performs required image processing, generation of display image data, or the like. The operating unit 117 outputs a command required for the main control unit 115 based on the operation performed by the user. In the present embodiment, the operating unit 117 is configured to include key switches. The display device 118 displays the display image data from the image processing unit 116. In the present embodiment, the display device 118 is configured to include a liquid crystal display. The control unit 131 of the processing circuit 130 may perform a part of the control performed by the main control unit 115, and the main control unit 115 may perform a part of the control performed by the control unit 131.

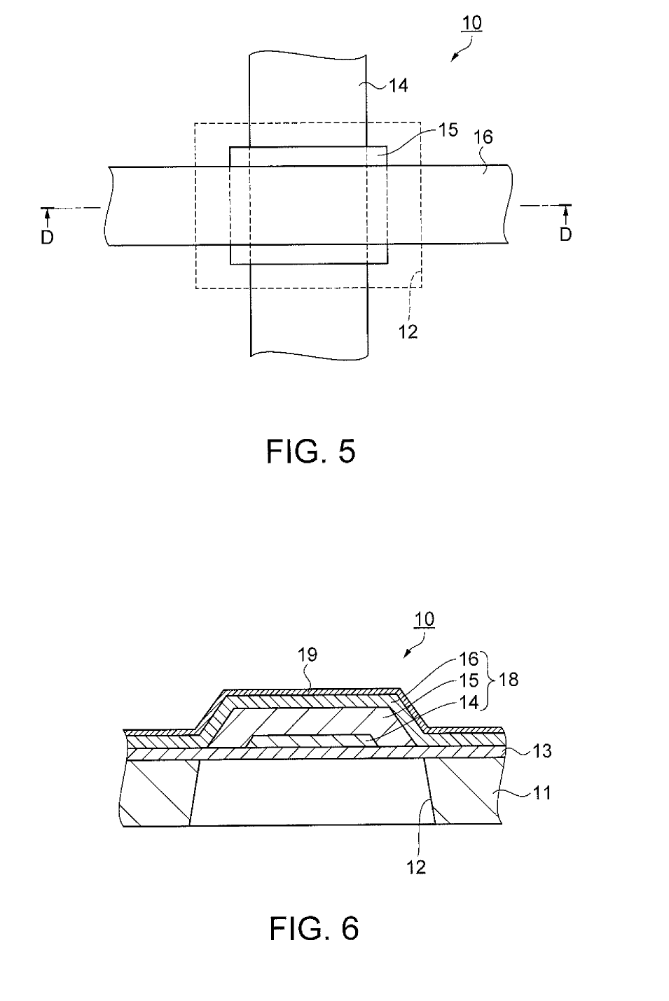

FIG. 5 is a plan view showing the schematic configuration of the ultrasonic element 10. FIG. 6 is a sectional view showing the schematic configuration of the ultrasonic element 10. In addition, FIG. 6 shows a section taken along the line D- of FIG. 5. The configuration of the ultrasonic element 10 of the present embodiment will be described with reference to FIGS. 5 and 6. The ultrasonic element 10 of the present embodiment is a thin film piezoelectric ultrasonic element.

As shown in FIGS. 5 and 6, the ultrasonic element 10 includes a base substrate 11, a vibrating film 13 formed on the base substrate 11, and a piezoelectric portion 18 provided on the vibrating film 13. The piezoelectric portion 18 includes a first electrode 14, a piezoelectric layer 15, and a second electrode 16.

In the ultrasonic element 10, an opening 12 is provided in the base substrate 11 formed of silicon or the like, and the vibrating film 13 is provided so as to cover the opening 12. The opening 12 is formed by etching, such as reactive ion etching (RIE), from the back surface (surface on which no element is formed) side of the base substrate 11. For example, the vibrating film 13 is formed as a two-layer structure including a silicon oxide (SiO₂) layer and a zirconium oxide (ZrO₂) layer. Here, in a case where the base substrate 11 is a silicon substrate, the silicon oxide layer can be formed by performing thermal oxidation processing on the substrate surface. The zirconium oxide layer is formed on the silicon oxide layer using a sputtering method, for example. Here, in the case of using, for example, lead zirconate titanate (PZT) as the piezoelectric layer 15 to be described later, the zirconium oxide layer is a layer for preventing the lead forming the PZT from diffusing into the silicon oxide layer. The zirconium oxide layer also has an effect of improving the deflection efficiency against the distortion of the piezoelectric layer 15.

The first electrode 14 is formed on the upper surface of the vibrating film 13, the piezoelectric layer 15 is formed on the upper surface of the first electrode 14, and the second electrode 16 is formed on the upper surface of the piezoelectric layer 15. In other words, the piezoelectric portion 18 is formed in a structure in which the piezoelectric layer 15 is interposed between the first electrode 14 and the second electrode 16.

In a case where the first electrode 14 is formed using a metal thin film and includes a plurality of ultrasonic elements 10 (piezoelectric layer 15), the first electrode 14 maybe a wiring line that extends to the outside of the element forming region to be connected to the ultrasonic element 10 adjacent thereto (piezoelectric layer 15), as shown in FIG. 5.

The piezoelectric layer 15 is formed using, for example, a lead zirconate titanate (PZT) thin film, and is provided so as to cover at least a part of the first electrode 14. The material of the piezoelectric layer 15 is not limited to the PZT. For example, lead titanate (PbTiO₃), lead zirconate (PbZrO₃), lead lanthanum titanate ((Pb, La) TiO₃) may be used.

The second electrode 16 is formed using a metal thin film, and is provided so as to cover at least a part of the piezoelectric layer 15. In a case where the second electrode includes a plurality of ultrasonic elements 10 (piezoelectric layer 15), the second electrode 16 may be a wiring line that extends to the outside of the element forming region to be connected to the ultrasonic element 10 adjacent thereto (piezoelectric layer 15), as shown in FIG. 5.

In addition, as shown in FIG. 6, a moisture-proof layer 19 that covers the ultrasonic element 10 in order to block moisture from the outside is provided. This moisture-proof layer 19 is formed of a material, such as alumina, and is provided on the entire surface of the ultrasonic element 10 or on a part thereof. The moisture-proof layer 19 may be appropriately set depending on the conditions or environment of use, or the moisture-proof layer 19 may not be provided.

The piezoelectric layer 15 expands and contracts in the in-plane direction when a voltage is applied between the first electrode 14 and the second electrode 16. Therefore, when a voltage is applied to the piezoelectric layer 15, for example, bending that causes to become convex on the opening 12 side occurs to deflect the vibrating film 13. By applying an AC voltage to the piezoelectric layer 15, the vibrating film 13 vibrates in a thickness direction, and ultrasonic waves are emitted from the opening 12 by the vibration of the vibrating film 13. In addition, ultrasonic waves are emitted to the opposite side (element formation side) to the opening 12. In addition, the ultrasonic device 1 of the present embodiment emits the ultrasonic waves, which are emitted to the opposite side (element formation side) to the opening 12, to the subject.

The ultrasonic element 10 also operates as a reception element that receives ultrasonic echoes, which return due to reflection on the object, of the emitted ultrasonic waves. The vibrating film 13 is vibrated by the ultrasonic echoes. Due to this vibration, stress is applied to the piezoelectric layer 15 to generate a voltage between the first electrode 14 and the second electrode 16. This voltage can be taken out as a reception signal.

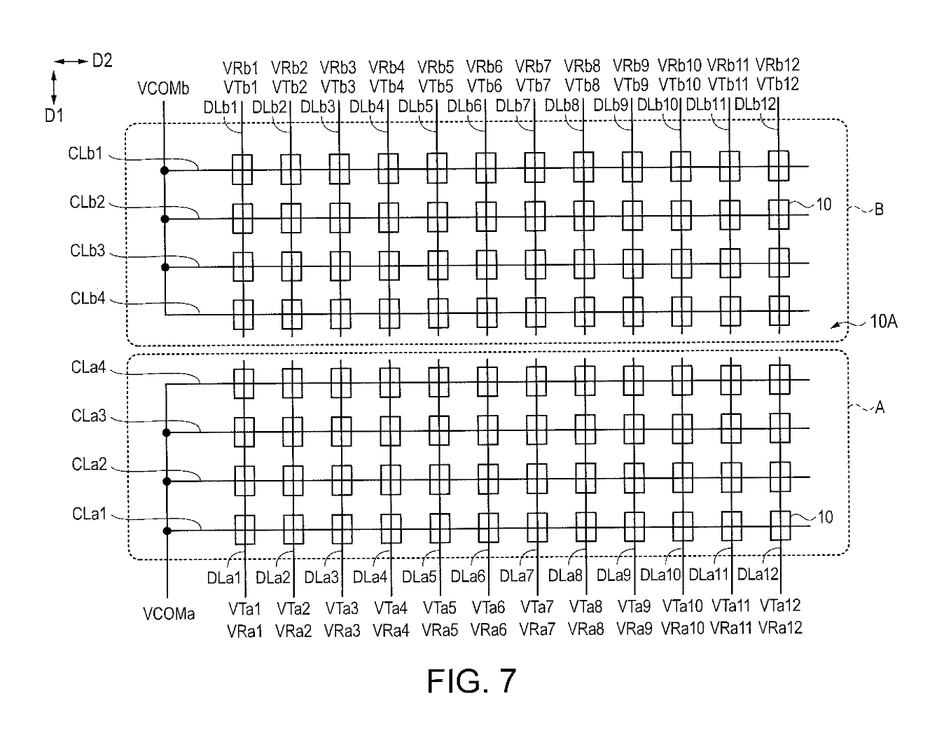

FIG. 7 is an explanatory view showing the schematic configuration of the ultrasonic element array 10A. The ultrasonic element array 10A in which the ultrasonic elements 10 described above are arranged in an array will be described with reference to FIG. 7.

The ultrasonic element array 10A is formed by arranging a plurality of ultrasonic elements 10 in an array. A plurality of ultrasonic elements 10 are arranged in a matrix of m rows and n columns. In FIG. 7, as an example, the ultrasonic element array 10A is configured such that eight rows of ultrasonic elements are arranged in the slice direction D1 and twelve columns of ultrasonic elements are arranged in the scan direction D2. The present embodiment will be described based on the ultrasonic element array 10A shown in FIG. 7.

The ultrasonic element array 10A of the present embodiment is configured to include the ultrasonic element array A corresponding to a first ultrasonic element array and the ultrasonic element array B corresponding to a second ultrasonic element array. In the present embodiment, therefore, the ultrasonic element array 10A is configured to include the two ultrasonic element arrays A and B. In both the ultrasonic element array A and the ultrasonic element array B, the ultrasonic elements 10 that form one channel (ultrasonic elements 10 connected to each other by a driving electrode line DL to be described later) are arranged in a first direction (corresponding to the scan direction D2 in the present embodiment). The ultrasonic element array B is disposed so as to be shifted from the ultrasonic element array A in a second direction (corresponding to the slice direction D1 in the present embodiment) crossing the first direction (approximately perpendicular to the first direction in the present embodiment).

Specifically, for the ultrasonic element array 10A configured in eight rows and twelve columns, both of the ultrasonic element array A and the ultrasonic element array B are configured to include regions of four rows and twelve columns. In other words, the ultrasonic element array A and the ultrasonic element array B of the present embodiment are configured by dividing the ultrasonic element array 10A into two parts in the slice direction D1.

The arrangement of the ultrasonic elements 10 is not limited to the matrix arrangement of eight rows and twelve columns. In addition, the ultrasonic element array A and the ultrasonic element array B are not limited to the configuration of four rows and twelve columns.

The ultrasonic element array 10A is configured to include not only the ultrasonic elements 10 but also driving electrode lines DLa and DLb and common electrode lines CLa and CLb. The ultrasonic element array A is configured to include the ultrasonic elements 10, the driving electrode line DLa, and the common electrode line CLa. The ultrasonic element array B is configured to include the ultrasonic elements 10, the driving electrode line DLb, and the common electrode line CLb.

By driving the ultrasonic element arrays A and B separately, each of the ultrasonic element arrays A and B can transmit and receive ultrasonic waves. In addition, by driving all of the ultrasonic element arrays A and B, the ultrasonic element arrays A and B can transmit and receive ultrasonic waves. In the present embodiment, since the ultrasonic element array 10A is configured to include the two ultrasonic element arrays A and B, it can be said that driving all of the ultrasonic element arrays A and B is driving the ultrasonic element arrays A and B in combination.

In the ultrasonic element array A, each driving electrode line DLa is wired along the slice direction D1. The respective driving electrode lines DLa are assumed to be driving electrode lines DLa1 to DLa12. Each channel is formed by a series of ultrasonic elements 10 connected to each other by each driving electrode line DLa. During a transmission period for which ultrasonic waves are transmitted, transmission signals VTa1 to VTa12 output from the processing circuit 130 forming the processing device 110 are supplied to the ultrasonic elements 10 through the driving electrode lines DLa1 to DLa12. In addition, during a reception period for which echo signals of the ultrasonic waves are received, reception signals VRa1 to VRa12 from the ultrasonic elements 10 are output to the processing circuit 130 through the driving electrode lines DLa1 to DLa12.

In the ultrasonic element array A, each common electrode line CLa is wired along the scan direction D2. The respective common electrode lines CLa are assumed to be common electrode lines CLa1 to CLa4. A common voltage VCOMa is supplied to the common electrode lines CLa1 to CLa4. The common voltage VCOMa may be a fixed DC voltage, and may not be 0 V, that is, a ground potential.

Also in the ultrasonic element array B, components are arranged approximately symmetrically in approximately the same manner as in the ultrasonic element array A. Specifically, in the ultrasonic element array B, each driving electrode line DLb is wired along the slice direction D1. The respective driving electrode lines DLb are assumed to be driving electrode lines DLb1 to DLb12. Each channel is formed by a series of ultrasonic elements 10 connected to each other by each driving electrode line DLb, similar to the ultrasonic element array A. During a transmission period for which ultrasonic waves are transmitted, transmission signals VTb1 to VTb12 output from the processing circuit 130 forming the processing device 110 are supplied to the ultrasonic elements 10 through the driving electrode lines DLb1 to DLb12. In addition, during a reception period for which echo signals of the ultrasonic waves are received, reception signals VRb1 to VRb12 from the ultrasonic element 10 are output to the processing circuit 130 through the driving electrode lines DLb1 to DLb12.

In the ultrasonic element array B, each common electrode line CLb is wired along the scan direction D2. The respective common electrode lines CLb are assumed to be common electrode lines CLb1 to CLb4. A common voltage VCOMb is supplied to the common electrode lines CLb1 to CLb4. The common voltage VCOMb may be a fixed DC voltage, and may not be a ground potential.

During the transmission period, a voltage corresponding to the difference between the transmission signal voltage and the common voltage is applied to each ultrasonic element 10, and an ultrasonic wave having a predetermined frequency is emitted. In the present embodiment, the voltage (driving voltage) applied to the ultrasonic element 10 is, for example, 10 V to 30 V (peak-to-peak value). The frequency is, for example, 1 MHz to 10 MHz.

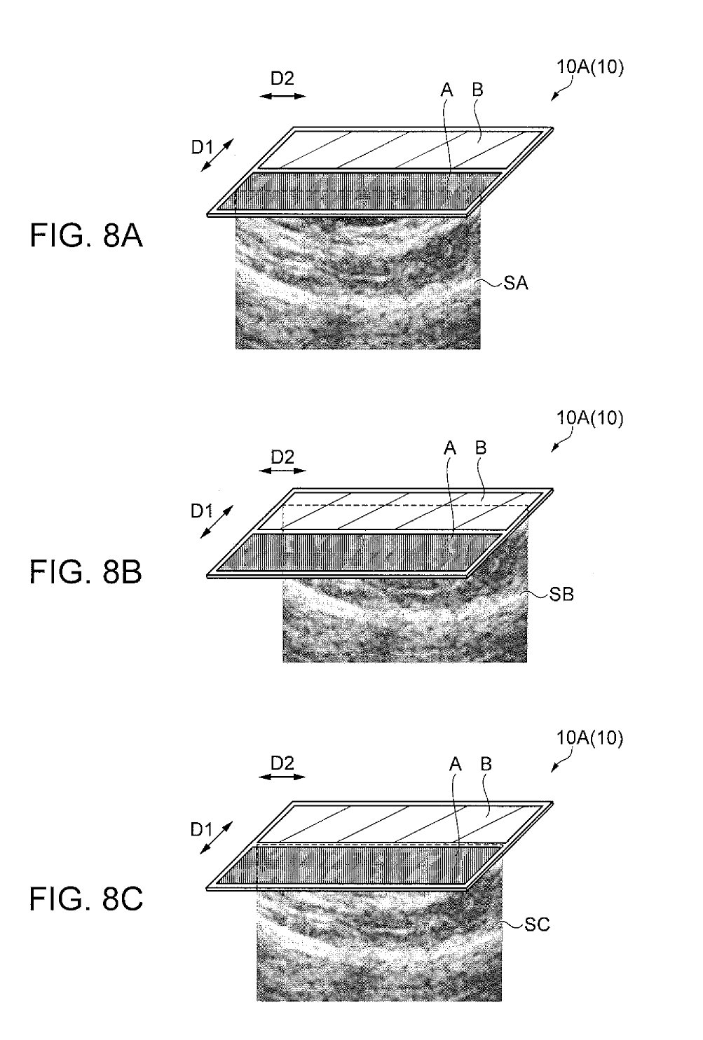

FIG. 8A is a schematic diagram showing a sectional image SA obtained in the case of driving the ultrasonic element array A. FIG. 8B is a schematic diagram showing a sectional image SB obtained in the case of driving the ultrasonic element array B. FIG. 8C is a schematic diagram showing a sectional image SC obtained in the case of driving both the two ultrasonic element arrays A and B. The relationship between each ultrasonic element array and the sectional image will be described with reference to FIGS. 8A to 8C. The sectional images SA, SB, and SC are shown as image views.

As described above, in the ultrasonic imaging apparatus 120 of the present embodiment, it is possible to make the ultrasonic device 1 (ultrasonic element array 10A) perform three kinds of driving including the separate driving of the ultrasonic element arrays A and B and the driving of all of the ultrasonic element arrays A and B. Then, the ultrasonic imaging apparatus 120 can generate three sectional images SA, SB, and SC by driving the ultrasonic element arrays A and B. Then, the ultrasonic imaging apparatus 120 of the present embodiment generates the sectional images SA, SB, and SC in the slice direction D1 (second direction) as B-mode display images, which are two-dimensional images, and displays generated sectional images SA, SB, and SC. In a case where a two-dimensional image is generated, a depth direction of the image corresponds to the slice direction D1 (second direction), and a left and right direction of the image corresponds to the scan direction D2 (first direction).

As shown in FIG. 8A, in the case of driving the ultrasonic element 10 of the ultrasonic element array A, ultrasonic waves are emitted from the ultrasonic element array A to be incident on the inside of the subject in contact with the ultrasonic element array A in a transmission period. Then, in a reception period, ultrasonic waves reflected from the inside of the subject are incident on the ultrasonic element array A as an echo signal. The incident echo signal is generated as the sectional image SA by the processing device 110. Thus, as the sectional image SA when driving the ultrasonic element array A, it is possible to obtain a sectional image in a direction immediately below the centerline in the slice direction D1 of the ultrasonic element array A.

As shown in FIG. 8B, also in the case of driving the ultrasonic element 10 of the ultrasonic element array B, ultrasonic waves are emitted from the ultrasonic element array B to be incident on the inside of the subject in contact with the ultrasonic element array B in a transmission period, in the same manner as in the case of driving the ultrasonic element 10 of the ultrasonic element array A. Then, in a reception period, ultrasonic waves reflected from the inside of the subject are incident on the ultrasonic element array B as an echo signal, the incident echo signal is generated as the sectional image SB by the processing device 110. Thus, as the sectional image SB when driving the ultrasonic element array B, it is possible to obtain a sectional image in a direction immediately below the centerline in the slice direction D1 of the ultrasonic element array B.

As shown in FIG. 8C, in the case of driving the ultrasonic elements 10 of the ultrasonic element arrays A and B, ultrasonic waves are emitted from the ultrasonic element arrays A and B to be incident on the inside of the subject in contact with the ultrasonic element arrays A and B in a transmission period. Then, in a reception period, ultrasonic waves reflected from the inside of the subject are incident on the ultrasonic element arrays A and B as an echo signal. The incident echo signal is generated as the sectional image SC by the processing device 110. Thus, as the sectional image SC when driving all of the ultrasonic element arrays A and B, it is possible to obtain a sectional image in a direction immediately below the centerline in the slice direction D1 of the ultrasonic element arrays A and B.

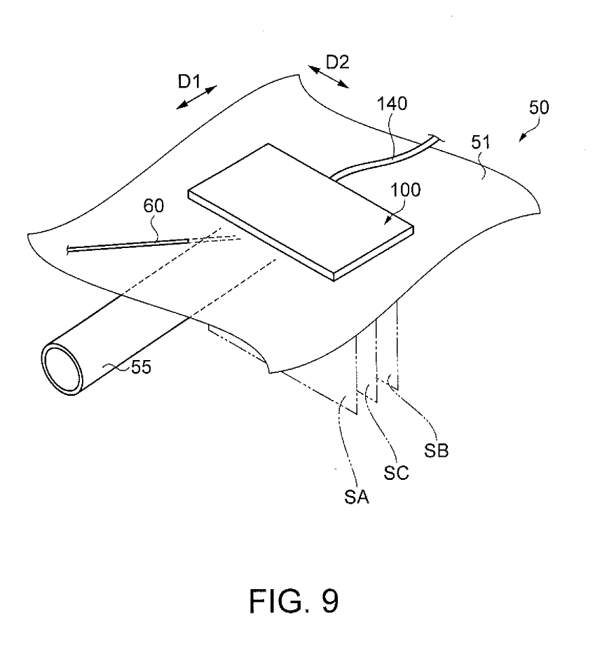

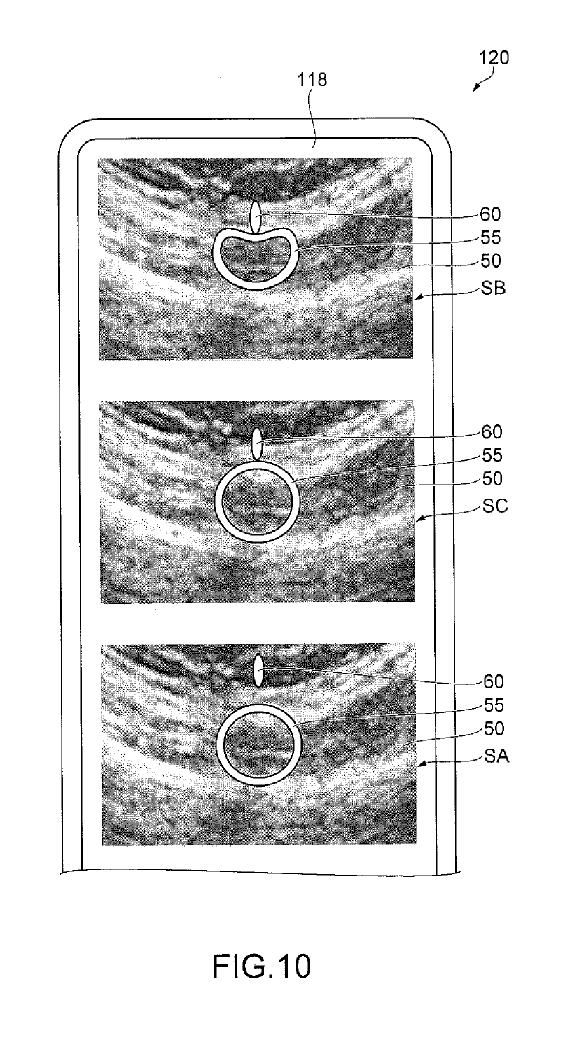

FIG. 9 is a schematic diagram showing the positional relationship among an insertion needle 60, a blood vessel 55, the ultrasonic probe 100, and the sectional images SA, SB, and SC in the case of performing an insertion operation. FIG. 10 is the sectional images SA, SB, and SC schematically showing a state of the insertion needle 60 at the time of insertion operation. The sectional images SA, SB, and SC shown in FIG. 10 are illustrated as image views. As an example, FIG. 10 shows a state in which the sectional images SA, SB, and SC are displayed on the display device 118 of the ultrasonic imaging apparatus 120. An approximate procedure in a case where an operator performs an insertion operation using the ultrasonic imaging apparatus 120 (ultrasonic probe 100) will be described with reference to FIGS. 9 and 10.

Hereinafter, a case of performing insertion into the blood vessel 55 of an arm 50, which is a subject, using the ultrasonic imaging apparatus 120 of the present embodiment will be described as an example.

As shown in FIG. 9, first, the ultrasonic probe 100 is placed on a skin surface 51 of the arm 50 so that a section of the blood vessel 55, into which the insertion needle 60 is to be inserted, is obtained as an image. Specifically, in order to obtain the section of the blood vessel 55, into which the insertion needle 60 is to be inserted, as an image, the ultrasonic probe 100 is placed such that the scan direction D2 is approximately perpendicular to a direction in which the blood vessel 55 extends. In other words, the ultrasonic probe 100 is placed such that the slice direction D1 is approximately parallel to the direction in which the blood vessel 55 extends. In addition, the ultrasonic probe 100 is placed at an approximate position of the skin surface 51, which is located above the position of the blood vessel 55 into which the insertion needle 60 is to be inserted (referred to as an insertion position), with a gel for ultrasonic waves being applied thereon.

After placing the ultrasonic probe 100, the ultrasonic imaging apparatus 120 is operated. In the present embodiment, the ultrasonic imaging apparatus 120 sequentially drives the ultrasonic element array A, the ultrasonic element array B, and the ultrasonic element arrays A and B. Accordingly, the sectional images SA, SB, and SC are obtained in the slice direction D1 (second direction).

As shown in FIG. 9, the positional relationship among the sectional images obtained by the operation of the ultrasonic imaging apparatus 120 is the sectional image SA obtained by driving the ultrasonic element array A first, the sectional image SC obtained by driving all of the ultrasonic element arrays A and B next, and the sectional image SB obtained by driving the ultrasonic element array B next, along the slice direction D1 from the insertion start position (distal side of the ultrasonic probe 100).

As described above, FIG. 10 shows a state in which the obtained sectional images SA, SC, and SB are displayed on the display device 118 of the ultrasonic imaging apparatus 120. In the present embodiment, the obtained sectional images are displayed in order of the sectional image SA, the sectional image SC, and the sectional image SB from the lower side to the upper side of the display device 118. In addition, the display order can be set by the user.

As shown in FIG. 9, the operator starts the insertion of the insertion needle 60 in the slice direction D1 toward the blood vessel 55 in the arm 50 from the skin surface 51 on the distal side (ultrasonic element array A side) of the ultrasonic probe 100. At this time, the operator starts the insertion toward an assumed insertion position below the ultrasonic probe 100.

In a case where the operator starts and advances the insertion of the insertion needle 60, if the insertion needle 60 is located at a sectional position where the sectional image SA is formed, the insertion needle 60 is reflected in the sectional image SA by the ultrasonic echo from the insertion needle 60 as shown in FIG. 10. If the operator advances the insertion further so that the insertion needle 60 is located at a sectional position where the sectional image SC is formed, the insertion needle 60 is similarly reflected in the sectional image SC. If the operator advances the insertion further so that the insertion needle 60 is located at a sectional position where the sectional image SB is formed, the insertion needle 60 is similarly reflected in the sectional image SB.

Here, assuming that the sectional image SB shown in FIG. 10 is an image at the moment that the needle tip of the insertion needle 60 is reflected, a state is shown in which the needle tip of the insertion needle 60 comes in contact with the blood vessel 55 to press the blood vessel 55. In this case, by advancing the insertion slightly from this state, the needle tip of the insertion needle 60 is inserted into the blood vessel 55. Accordingly, the insertion of the insertion needle 60 is completed.

After placing the ultrasonic imaging apparatus 120 on the skin surface 51 as described above, the operator performs the insertion operation in the slice direction D1 while checking a sectional image in which the needle tip is reflected. Therefore, the operator can perform the insertion operation reliably and safely without losing the sight of the needle tip.

According to the embodiment described above, the following effects are obtained.

In the ultrasonic device 1 of the present embodiment, the ultrasonic element array 10A is configured to include the two ultrasonic element arrays A and B. The ultrasonic element array A corresponds to the first ultrasonic element array in which the ultrasonic elements 10 forming one channel are arranged in the first direction (corresponding to the scan direction D2 in the present embodiment). The ultrasonic element array B corresponds to the second ultrasonic element array in which the ultrasonic elements 10 forming one channel are arranged in the first direction. The second ultrasonic element array is disposed so as to be shifted from the first ultrasonic element array in the second direction (corresponding to the slice direction D1 in the present embodiment) crossing the first direction. In addition, it is possible to transmit and receive ultrasonic waves by driving all of the ultrasonic element arrays A and B in addition to the separate driving of the ultrasonic element arrays A and B. Accordingly, it is possible to receive ultrasonic echoes for generating the three sectional images SA, SB, and SC in the second direction (slice direction D1). As a result, it is possible to realize the ultrasonic device 1 capable of easily capturing the needle tip of the insertion needle 60 with the simplest configuration.

According to the ultrasonic probe 100 of the present embodiment, the ultrasonic probe 100 is formed by housing the ultrasonic device 1 including the two ultrasonic element arrays A and B in the housing member 80. Therefore, it is possible to realize the ultrasonic probe 100 capable of easily capturing the needle tip without moving the ultrasonic probe 100.

According to the ultrasonic imaging apparatus 120 of the present embodiment, the three sectional images SA, SB, and SC are generated in the second direction (slice direction D1) by driving the two ultrasonic element arrays A and B. Therefore, it is possible to realize the ultrasonic imaging apparatus 120 capable of easily capturing the needle tip without moving the ultrasonic probe 100. In addition, by using the ultrasonic imaging apparatus 120 configured as described above, the operator can perform the insertion operation reliably and safely without losing the sight of the needle tip.

The ultrasonic device 1, the ultrasonic probe 100, and the ultrasonic imaging apparatus 120 of the present embodiment can be suitably used for nerve block therapy, biopsy, radiofrequency ablation therapy (RFA), blood sampling, carotid echo inspection, and the like.

### Second Embodiment

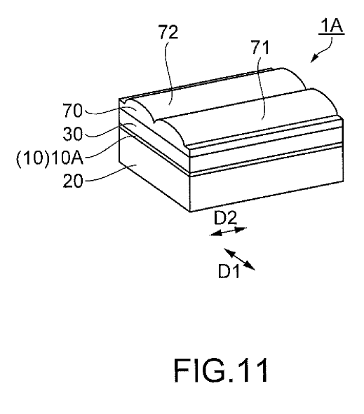

FIG. 11 is a perspective view showing the schematic configuration of an ultrasonic device 1A according to a second embodiment. The configuration and operation of the ultrasonic device 1A of the present embodiment will be described with reference to FIG. 11.

In the ultrasonic device 1A of the present embodiment, the configuration of an acoustic unit 70 is different from that in the ultrasonic device 1 of the first embodiment. The other configuration is the same as the ultrasonic device 1 of the first embodiment. The same components as in the first embodiment are denoted by the same reference numerals.

In the acoustic unit 70 of the present embodiment, the flat surface portion 41 of the acoustic unit 40 of the first embodiment is replaced with two convex lenses. The two lenses are assumed to be lenses 71 and 72. Accordingly, the acoustic unit 70 is configured to include the lenses 71 and 72. The lens 71 is formed corresponding to the ultrasonic element array A, and the lens 72 is formed corresponding to the ultrasonic element array B. Each of the lenses 71 and 72 is formed in a convex and partially cylindrical shape, and is formed so as to extend in the scan direction D2. The curvature of each of the lenses 71 and 72 is set according to the focal position of ultrasonic waves.

The ultrasonic imaging apparatus 120 configured to include the ultrasonic device 1A of the present embodiment instead of the ultrasonic device 1 of the first embodiment performs only the separate driving of the ultrasonic element arrays A and B. In the ultrasonic imaging apparatus 120 of the present embodiment, therefore, the driving of all of the ultrasonic element arrays A and B is not performed.

By driving the ultrasonic device 1A using the ultrasonic imaging apparatus 120 of the present embodiment, it is possible to obtain two sectional images of a sectional image corresponding to the ultrasonic element array A (corresponding to the sectional image SA in the first embodiment) and a sectional image corresponding to the ultrasonic element array B (corresponding to the sectional image SB in the first embodiment).

According to the embodiment described above, the following effects can be achieved in addition to the same effect as in a case where driving all of the ultrasonic element arrays A and B in the first embodiment is excluded.

According to the ultrasonic device 1A of the present embodiment, the acoustic unit 70 includes the lenses 71 and 72 corresponding to the ultrasonic element arrays A and B. Therefore, since the spread of ultrasonic waves emitted from the ultrasonic element array A can converge further compared with the first embodiment, it is possible to improve the resolution in the ultrasonic element array A. This is the same for the ultrasonic element array B. Accordingly, it is possible to improve the resolution of a sectional image displayed on the display device 118 of the ultrasonic imaging apparatus 120 compared with the first embodiment.

The invention is not limited to the embodiments described above, and can be implemented by adding various modifications, improvements, or the like within a range not departing from the spirit of the invention. Modification examples will be described below.

The ultrasonic element array 10A of the first embodiment is configured to include the ultrasonic element array A as a first ultrasonic element array and the ultrasonic element array B as a second ultrasonic element array. Specifically, the ultrasonic element array 10A is configured to include the two ultrasonic element arrays A and B. However, without being limited thereto, the ultrasonic element array 10A may also be configured to include three or more ultrasonic element arrays that are formed so as to maintain the relationship between the first ultrasonic element array and the second ultrasonic element array. In this case, the ultrasonic device 1 and the ultrasonic probe 100 can perform transmission and reception of ultrasonic waves for the three or more ultrasonic element arrays not only by driving ultrasonic element arrays separately but also by driving the ultrasonic element arrays in combination. Accordingly, since the ultrasonic imaging apparatus 120 can generate a larger number of sectional images than the number of sectional images in the first embodiment in the second direction, it is possible to capture the needle tip more easily without moving the ultrasonic device 1. This is the same in the second embodiment.

Driving the ultrasonic element arrays in combination includes driving two adjacent ultrasonic element arrays among a plurality of ultrasonic element arrays in combination, driving three or more adjacent ultrasonic element arrays in combination, driving ultrasonic element arrays, which are not adjacent to each other, in combination, and the like. In any case, in order to obtain sectional images required to easily capture the needle tip, effective ultrasonic element arrays may be driven in combination.

The ultrasonic imaging apparatus 120 of the first embodiment generates the three sectional images SA, SB, and SC. However, in a case where three sectional images are not required, an intended sectional image can be obtained by driving the required ultrasonic element arrays A and B by operating the operating unit 117.

In the ultrasonic imaging apparatus 120 of the second embodiment, the acoustic unit 70 includes the two lenses 71 and 72 corresponding to the two ultrasonic element arrays A and B. However, also in a case where three or more ultrasonic element arrays are provided, lenses corresponding to the number of ultrasonic element arrays may be provided. In this case, only the separate driving of ultrasonic element arrays maybe performed without driving the ultrasonic element arrays in combination.

What is claimed is:

1. An ultrasonic device having ultrasonic elements to transmit and receive ultrasonic waves, comprising:
a first ultrasonic element array in which ultrasonic elements forming one channel among the ultrasonic elements are arranged in a first direction; and
a second ultrasonic element array in which ultrasonic elements forming one channel are arranged in the first direction,
wherein the second ultrasonic element array is disposed so as to be shifted from the first ultrasonic element array in a second direction crossing the first direction.

2. The ultrasonic device according to claim 1,
wherein the first and second ultrasonic element arrays transmit and receive the ultrasonic waves by driving the first and second ultrasonic element arrays in combination in addition to driving the first and second ultrasonic element arrays separately.

3. The ultrasonic device according to claim 1,
wherein the first and second ultrasonic element arrays transmit and receive the ultrasonic waves by driving all of the ultrasonic elements of the first and second ultrasonic element arrays in addition to driving the first and second ultrasonic element arrays separately.

4. The ultrasonic device according to claim 1, further comprising:
an acoustic unit in contact with a subject,
wherein the acoustic unit includes a flat surface portion in contact with the subject.

5. An ultrasonic probe comprising:
the ultrasonic device according to claim 1; and
a housing member that houses the ultrasonic device such that a part of the ultrasonic device is exposed.

6. An ultrasonic probe comprising:
the ultrasonic device according to claim 2; and
a housing member that houses the ultrasonic device such that a part of the ultrasonic device is exposed.

7. An ultrasonic probe comprising:
the ultrasonic device according to claim 3; and
a housing member that houses the ultrasonic device such that a part of the ultrasonic device is exposed.

8. An ultrasonic probe comprising:
the ultrasonic device according to claim 4; and
a housing member that houses the ultrasonic device such that a part of the ultrasonic device is exposed.

9. An ultrasonic imaging apparatus comprising:
the ultrasonic probe according to claim 5;
a processing device that controls the ultrasonic probe and generates an image in the ultrasonic element array based on an input signal from the ultrasonic probe; and
a display device that displays the image generated by the processing device.

10. An ultrasonic imaging apparatus comprising:
the ultrasonic probe according to claim 6;
a processing device that controls the ultrasonic probe and generates an image in the ultrasonic element array based on an input signal from the ultrasonic probe; and
a display device that displays the image generated by the processing device.

11. An ultrasonic imaging apparatus comprising:
the ultrasonic probe according to claim 7;
a processing device that controls the ultrasonic probe and generates an image in the ultrasonic element array based on an input signal from the ultrasonic probe; and
a display device that displays the image generated by the processing device.

12. An ultrasonic imaging apparatus comprising;
the ultrasonic probe according to claim 8;
a processing device that controls the ultrasonic probe and generates an image in the ultrasonic element array based on an input signal from the ultrasonic probe; and
a display device that displays the image generated by the processing device.
